(19) United States
(12) Patent Application Publication
(10) Pub. No.: US 2025/0352335 A1
(43) Pub. Date: Nov. 20, 2025

(54) ANTENNAE FOR STENT AND HEART VALVES

(71) Applicant: EDWARDS LIFESCIENCES CORPORATION, Irvine, CA (US)

(72) Inventors: Blake W. Axelrod, South Pasadena, CA (US); Laxmi Ray, Costa Mesa, CA (US); Julia Leigh Ross, Huntington Beach, CA (US)

(21) Appl. No.: 19/285,198

(22) Filed: Jul. 30, 2025

Related U.S. Application Data
(63) Continuation of application No. PCT/US2024/013411, filed on Jan. 29, 2024.
(60) Provisional application No. 63/482,199, filed on Jan. 30, 2023.

(51) Int. Cl. A61F 2/24; A61B 5/00; A61B 5/0215; H01Q 7/00

(52) U.S. Cl. CPC A61F 2/2418; A61B 5/0031; A61B 5/0215; A61B 5/6862; H01Q 7/00

(57) ABSTRACT

A prosthetic valve includes a flexible frame disposed along and deformable about a frame axis. The frame includes a first end, a second end oppositely disposed from the first end, a plurality of post assemblies extending axially away from the first end, and a network of interconnected struts defining a plurality of cells. A circuit is disposed axially away from the frame and mounted on the plurality of post assemblies. The circuit includes an antenna coil secured to at least two post assemblies of the plurality of post assemblies, and a sensor in electrical communication with the antenna coil. The sensor is configured to sense a physical parameter and generate a signal representing the physical parameter.

### CROSS-REFERENCE TO RELATED APPLICATION(S)

This application is a continuation of International Application No. PCT/US2024/013411, filed Jan. 29, 2024, which claims the benefit of U.S. Provisional Application No. 63/482,199, filed Jan. 30, 2023, the disclosures of which are hereby incorporated by reference in their entireties.

### BACKGROUND

The present disclosure relates to implantable medical devices, and in particular, to implantable prosthetic valves.

Various medical procedures involve the implantation of medical devices within the anatomy of the heart. Certain physiological parameters associated with such anatomy, such as fluid pressure, can have an impact on patient health prospects. Accordingly, systems, devices and methods for post-operatively monitoring recipients of medical implant devices, including in an environment outside of the hospital or care facility, are desirable for improving patient outcomes.

### SUMMARY

In one example, a prosthetic valve includes a flexible frame disposed along and deformable about a frame axis. The frame includes a first end, a second end oppositely disposed from the first end, a plurality of post assemblies extending axially away from the first end, and a network of interconnected struts defining a plurality of cells. A circuit is disposed axially away from the frame and mounted on the plurality of post assemblies. The circuit includes an antenna coil secured to at least two post assemblies of the plurality of post assemblies, and a sensor in electrical communication with the antenna coil. The sensor is configured to sense a physical parameter and generate a signal representing the physical parameter.

In another example, a prosthetic valve includes a flexible frame disposed along and deformable about a frame axis. The frame includes a first end, a second end oppositely disposed from the first end, a plurality of post assemblies extending axially away from the first end, and a network of interconnected struts defining a plurality of cells. A circuit is disposed axially away from the frame and mounted on the plurality of post assemblies. The circuit includes an antenna coil secured to at least two post assemblies of the plurality of post assemblies, and a sensor in electrical communication with the antenna coil. A plurality of hollows sleeves circumscribe the antenna coil. The sensor is configured to sense a physical parameter and generate a signal representing the physical parameter.

### BRIEF DESCRIPTION OF THE DRAWINGS

FIG. 1 is a partial cross-sectional schematic of the heart of with the four chambers shown in cross-section.

FIG. 2 is a block diagram representing a monitoring system for monitoring one or more physiological parameters associated with a patient.

FIG. 3 is a perspective view of a first example of a prosthetic heart valve shown in an expanded state.

FIG. 4 is a perspective view of the prosthetic heart valve of FIG. 3 shown in a crimped state.

FIG. 5 is a block diagram representing select components of an active sensing circuit of the prosthetic heart valve.

FIG. 6 is a simplified illustration of a second example of a prosthetic heart valve shown in the expanded state.

FIG. 7 is simplified illustration of a sensing circuit of the prosthetic heart valve of FIG. 6 shown in the crimped state.

FIG. 8 is a simplified illustration of a third example of a prosthetic heart valve shown in the expanded state.

FIG. 9 is a simplified illustration of a sensing circuit of the prosthetic heart valve of FIG. 8 shown in the crimped state.

### DETAILED DESCRIPTION

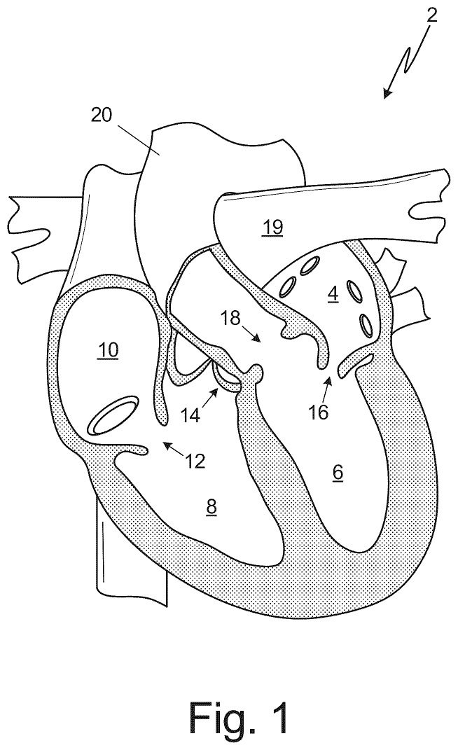

FIG. 1 is a partial cross-sectional schematic of heart 2, which in one example, belongs to a human patient. Heart 2 includes four chambers, including left atrium 4, left ventricle 6, right ventricle 8, and right atrium 10. The four chambers are shown in cross-section in FIG. 1. Heart 2 further includes four valves for aiding the circulation of blood therein, including tricuspid valve 12, pulmonary valve 14, mitral valve 16, and aortic valve 18. FIG. 1 further shows pulmonary artery 19 and aorta 20.

Tricuspid valve 12 separates right atrium 10 from right ventricle 8 and can include three cusps or leaflets. Tricuspid valve 12 can close during ventricular contraction (i.e., systole) and open during ventricular expansion (i.e., diastole). Pulmonary valve 14 separates right ventricle 8 from pulmonary artery 19 and may be configured to open during systole so that blood may be pumped towards the lungs, and close during diastole to prevent blood from leaking back into heart 2 from pulmonary artery 19. Similar to tricuspid valve 12, pulmonary valve 14 can have three cusps/leaflets, each one resembling a crescent. Mitral valve 16 separates left atrium 4 from left ventricle 6 and can have two cusps or leaflets. Mitral valve 16 is configured to open during diastole so that blood in left atrium 4 can flow into left ventricle 6, and close during systole to prevent blood from leaking back into left atrium 4. Aortic valve 18 separates left ventricle 6 from aorta 20. Aortic valve 18 is configured to open during systole to allow blood leaving left ventricle 6 to enter aorta 20, and close during diastole to prevent blood from leaking back into left ventricle 6.

A heart valve can include a relatively dense fibrous ring, referred to herein as the annulus, as well as a plurality of leaflets or cusps attached to the annulus. Some valves can further include a collection of chordae tendineae and papillary muscles securing the leaflets. Generally, the size of the leaflets or cusps may be such that when the heart contracts, the resulting increased blood pressure produced within the corresponding heart chamber forces the leaflets open at least partially to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

Heart valve disease represents a condition in which one or more of the valves of heart 2 fail to function properly. Diseased heart valves may be categorized as stenotic, wherein the valve does not open sufficiently to allow adequate forward flow of blood through the valve, and/or incompetent, wherein the valve does not close completely, causing excessive backward flow of blood through the valve when the valve is closed. In certain conditions, valve disease can be severely debilitating and even fatal if left untreated.

To treat disease of a heart valve, a prosthetic heart valve can be implanted in and sutured to the respective valve annulus. Such a prosthetic heart valve can be positioned with its openings oriented in the direction of blood flow through the valve. The prosthetic heart valve can be configured to operate as the diseased valve it is replacing such that it can allow unidirectional blood flow through the valve while preventing flow in the reverse direction.

In a typical cardiac implant procedure, the heart can be incised, and in a valve replacement operation, the defective valve can be removed leaving the desired placement site that can include the valve annulus. Sutures can be passed through fibrous tissue of the annulus or desired placement site to form an array of sutures. Free ends of the sutures may be individually threaded through a suture-permeable sealing edge of the prosthetic heart valve. Artificial heart valves can be used to replace faulty or deteriorating natural heart valves in patients with heart valve disorders including aortic stenosis, mitral regurgitation, etc. The valve replacement process generally involves surgical or transcatheter procedures (e.g., balloon valvotomy) to replace the existing valves with the new artificial valves. Since the artificial valves are a foreign body, many different challenges and issues can be involved with such a procedure. For example, paravalvular leakage (PVL) and/or leaflet thickening can occur in patients who undergo heart valve replacement. Similarly, rejection of an artificial surgical heart valve due to thrombus can occur, requiring the patient to use anti-coagulants for proper valve operation.

Some methods for monitoring valve performance after implantation involve using complex bio-imaging techniques, such as echocardiography. Such methods can generally only be performed in specialized medical facilities and can cost significant time and money. Hence, such methods may generally only be used once symptoms of valve malfunction are detected. Some artificial valves may not provide the ability to detect changes in operation to detect problems early on. Moreover, many patients who suffer from valvular disease and require an artificial valve may also suffer from other cardiovascular disorders, including heart failure. Some artificial heart valve systems may not allow for gathering data about the valve and/or the patient's condition postoperatively in an outpatient setting (e.g., a cardiologist visit in a ward) using existing patient monitoring systems. Such systems may not provide for routine collection of data at sufficient resolution to enable development of new digital solutions for better management of the patients as their numbers and diversity increase over time.

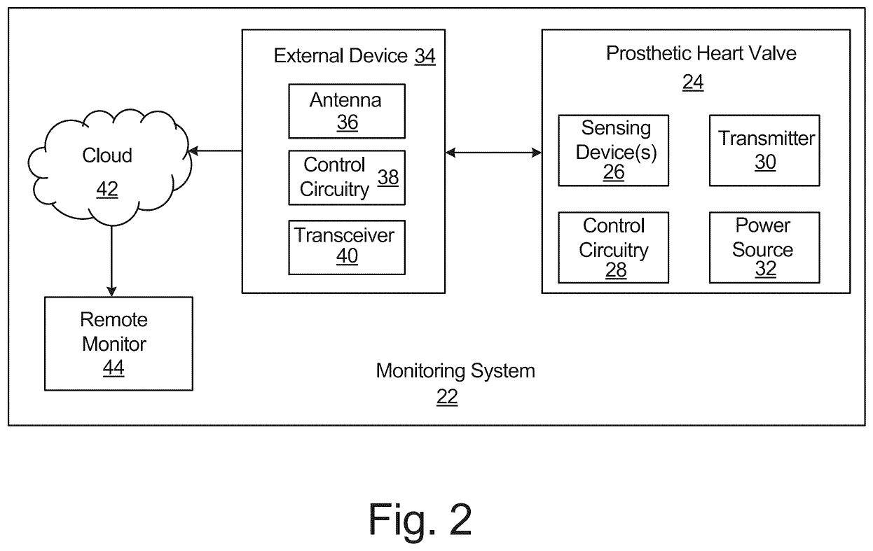

Accordingly, a prosthetic heart valve can be part of a larger system for post-operatively monitoring a patient, as will be discussed in reference to FIG. 2.

FIG. 2 is a block diagram representing monitoring system 22 for monitoring one or more physiological parameters associated with a patient. System 22 includes prosthetic heart valve 24, which includes sensing devices 26, control circuitry 28, transmitter 30, and power source 32. System 22 further includes external device 34, which includes antenna 36, control circuitry 38, and transceiver 40. System 22 also includes cloud 42 and remote monitor 44.

Prosthetic heart valve 24 can include one or more sensing devices 26, control circuitry 28, transmitter 30, and power source 32. Sensing devices 28 can include one or more of following types of sensors/transducers: MEMS sensors, optical sensors, piezoelectric sensors, electromagnetic sensors, strain sensors/gauges, accelerometers, gyroscopes, and/or other types of sensors, which can be positioned in the patient to sense one or more parameters relevant to the health of the patient. Control circuitry 28 can be wired or wirelessly connected to sensing devices 26 and can include one or more of application-specific integrated circuit (ASIC), microcontrollers, chips, tuning capacitors, etc. Control circuitry 28 can receive signals from external device 34 (e.g., requests for stored or immediately acquired data), request data from sensors 26, and coordinate data transmission. Transmitter 30 can be, for example, an antenna for radiating an electronic signal transmitted by control circuitry 28. Power source 32 can be a suitable source of power able to minimize interference with the heart or other anatomy of the patient. In one example, power source 32 can be a passive means for wirelessly receiving external power (e.g., short-range or near-field wireless power transmission). In another example, power source 32 can be a battery, or a means for locally harvesting energy from within the patient.

External device 34, located at least partially outside of the patient, can be in wireless communication with prosthetic heart valve 24. External device 34 includes antenna 36, control circuitry 38, and transceiver 40. Antenna 36 can receive wireless signal transmissions from prosthetic heart valve 24. In one example, antenna 36 can be externally mounted to external device 34. Control circuitry 38 can be a processor or other suitable means for processing signals received from prosthetic heart valve 24. Transceiver 40 can be configured to receive and amplify signals from prosthetic heart valve 24, as well as to transmit signals to cloud 42 and remote monitor 44. Such signals can include, for example, pressure data acquired from sensors 26. Transceiver 40 can, accordingly, include one or more of digital-to-analog converter (DAC) circuitry, power amplifiers, low-pass filters, antenna switch modules, antennas, etc. for treatment and/or processing of transmitted and received signals.

External device 34 can serve as an intermediate communication device between prosthetic heart valve 24 and remote monitor 44. External device 34 can be a dedicated external unit designed to communicate with prosthetic heart valve 24. For example, external device 34 can be a wearable communication device, or another device that can be readily disposed in proximity to the patient and/or prosthetic heart valve 24. External device 34 can be configured to interrogate prosthetic heart valve 24 continuously, periodically, or sporadically in order to extract or request sensor-based information therefrom. In some examples, external device 34 can include a user interface upon which a user (e.g., the patient) can view sensor data, request sensor data, or otherwise interact with external device 34 and/or prosthetic heart valve 24.

Cloud 42 can be a secure network in communication with external device 34 via ethernet, Wi-Fi, or other network protocol. Cloud 42 can also be configured to implement data storage. In another example, cloud 42 can instead be a secure physical network. Remote monitor 44 can be in communication with external device 34 via cloud 42. Remote monitor 44 can be any type of computing device or collection of computing devices configured to receive, process and/or present monitor data received via cloud 42 from external device 34 or prosthetic heart valve 24. For example, remote monitor 44 can advantageously be operated and/or controlled by a healthcare entity, such as a hospital, doctor, or other care entity associated with the patient. Although certain examples disclosed herein describe communication with remote monitor 44 from prosthetic heart valve 24 indirectly through external device 34, prosthetic heart valve 24 can instead include a transmitter (e.g., transmitter 30) capable of communicating, via cloud 42, with remote monitor 44 without the necessity of relaying information through device 34.

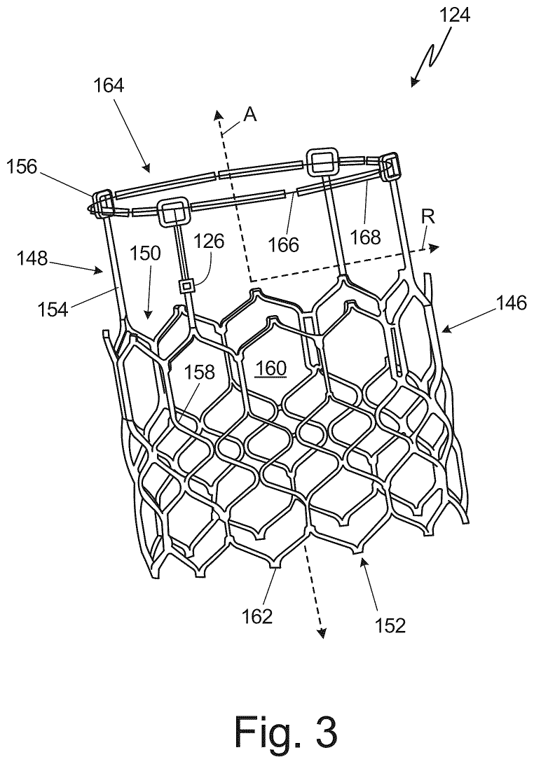

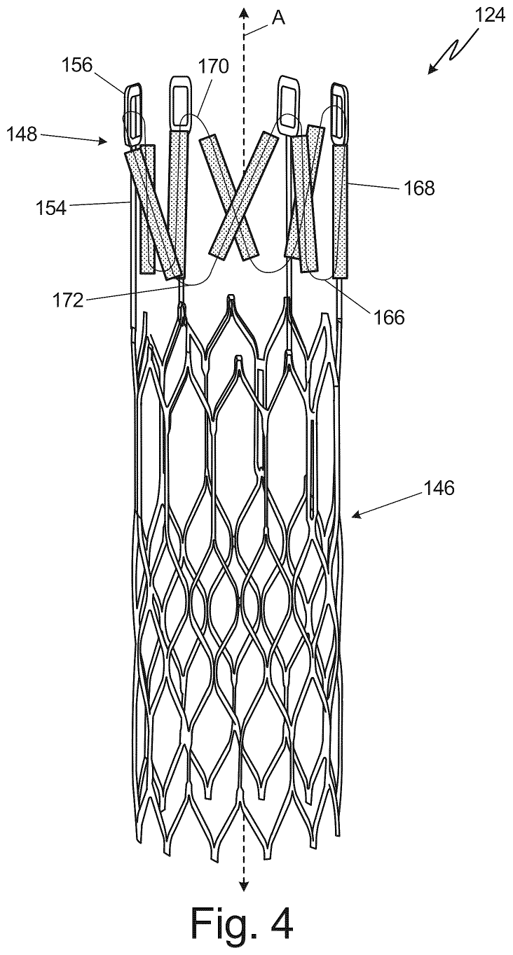

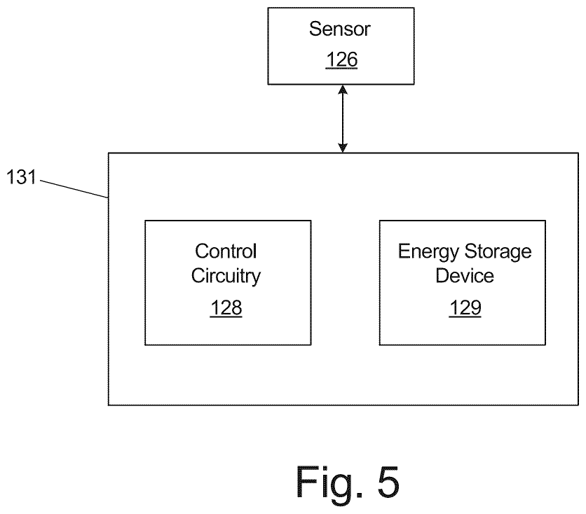

FIG. 3 is a perspective view of prosthetic heart valve 124, shown in an expanded state. FIG. 4 is a perspective view of prosthetic heart valve 124, shown in a crimped state. FIG. 5 is a block diagram representing one example of a sensing circuit of prosthetic heart valve 124. FIGS. 3, 4, and 5 are discussed together.

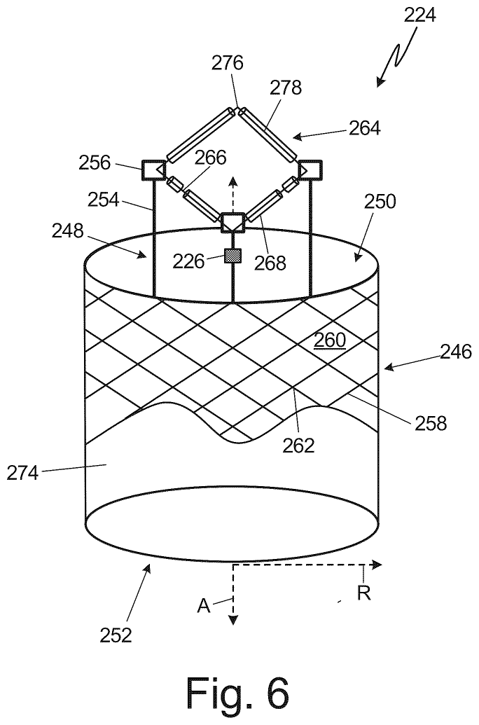

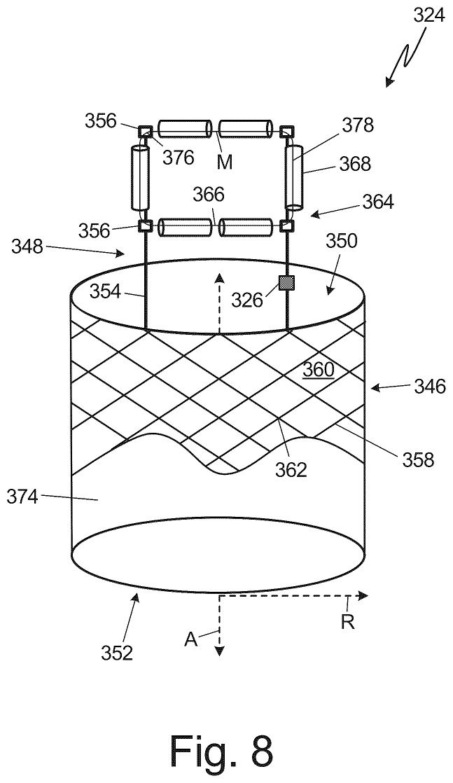

As shown in FIG. 3, structural components of prosthetic heart valve 124 include deformable frame 146 and post assemblies 148 extending axially away from frame 146 relative to valve axis A. Axis A can generally be aligned with the direction of blood flow through prosthetic heart valve 124 when implanted in a patient. Frame 146 and post assemblies 148 can be formed from a biocompatible metallic material (e.g., Nitinol). As shown in FIG. 3, four post assemblies 148 extend from top/upper end 150 of prosthetic heart valve 124 based on the orientation of FIG. 3. Post assemblies 148 can extend from bottom/lower end 152 in an alternative example. Each post assembly 148 can include post 154 and islet 156. Each post assembly can be about 1 cm in length. Sensor 126 can be mounted to one post 154. Frame 146 can include a network of struts 158 defining open cells 160 therebetween. Each cell 160 can include oppositely axially disposed pointed tips/ends 162. Frame 146 can be at least partially covered with a biocompatible fabric in another example, such as is shown and discussed with respect to FIGS. 6 and 8 below.

Prosthetic heart valve 124 further includes sensing circuit 164 for monitoring physiological parameters of a patient. Sensing circuit 164 includes deformable antenna coil 166 and sensor 126 electrically connected (e.g., via leads/wires) to antenna coil 166. Antenna coil 166 can include one or more individual wires formed from a conductive, but biocompatible, metallic material such as gold. Other examples can include copper or titanium. Antenna coil 166 can further be coated with an insulating material (e.g., silicone, parylene, or polyimide). A plurality of hollow sleeves 168 circumscribe antenna coil 166, with gaps between adjacent sleeves 168 such that antenna coil 166 is not completed circumscribed by sleeves 168. Sleeves 168 can be formed from a rigid polymer material, and the dimensions of each sleeve 168 (e.g., length, diameter, thickness, etc.) can be identical or can vary depending on the example. Antenna coil 166 can be secured to post assemblies 148 using biocompatible attachment means (e.g., sutures). Antenna coil 166 can be threaded through/around islets 156, as is depicted in FIG. 3, and sutured thereto, or looped around/sutured to posts 154 in another example. Sensor 126 can be a capacitive pressure sensor in one example, including a diaphragm and pressure cavity to form a variable capacitor to detect strain due to pressure applied to the diaphragm. In general, the capacitance of sensor 126 decreases as pressure deforms the diaphragm.

In the expanded state illustrated in FIG. 3, prosthetic heart valve 124 has an axial dimension or length, extending along axis A, and a radial dimension R extending radially outward from axis A. Antenna coil 166 is disposed about post assemblies 148, and can therefore assume a generally circular shape with a radial dimension based on R. Depending on the position of post assemblies 148, antenna coil 166 can include one or a combination of straight and/or curved segments in the expanded state, such that the shape need not be circular to operate as intended. The entire length of antenna coil 166 is generally equidistant from frame 146 in the expanded state. The diameter of prosthetic heart valve 124 and/or antenna coil 166 can be determined from the radial dimension (i.e., diameter=2(R)). In the crimped state illustrated in FIG. 4, prosthetic heart valve 124 has a relatively greater axial dimension, and a relatively smaller radial dimension, compared to the expanded state dimensions shown in FIG. 3. In the crimped state, antenna coil 166 deforms from a planar circular shape with almost no axial extent, to having a reduced diameter and an axially-disposed zig-zag pattern with peaks 170 and troughs 172. Peaks 170 and troughs 172 generally correspond with gaps between adjacent sleeves 168, and sleeves 168 are disposed between peaks 170 and troughs 172. This occurs because the relatively rigid sleeves 168 prevent bending of the encircled length of antenna coil 166, while the more flexible uncovered/exposed portions of antenna coil 166 at the gaps are permitted to deform during crimping. Thus, the number and placement of sleeves 168 can influence the movement and resulting pattern of antenna coil 166 during crimping. When transitioned from the crimped state to the expanded state, the radial expansion of prosthetic heart valve 124 allows sleeves 168 to straighten in the radial direction until peaks 170 and troughs 172 disappear as antenna coil 166 comes to rest in a single plane. Sutures secure antenna coil 166 to limit unwanted movement of antenna coil 166 and sleeves 168 so that antenna coil 166 more reliably transitions between the expanded and crimped states. The relative rigidity of sleeves 168 can help maintain the shape of antenna coil 166 in the expanded state, especially as prosthetic heart valve 124 experiences forces related to contraction and relaxation of the heart. This preserves the integrity of antenna coil 166 and its associated sensing circuit 164.

In one example, sensor 126 can be incorporated into an active sensing circuit, as shown schematically in FIG. 5. More specifically, sensor 126 can be in communication with control circuitry 128 and energy storage device 129 (e.g., a capacitor or battery). Control circuitry 128 and energy storage device 129 can be housed in container 131, which can be formed from a biocompatible material and hermetically sealed to prevent exposure to surrounding tissue. Sensor 126 can be closely associated with container 131 (e.g., as a deformable membrane) but need not be sealed inside to permit probing of the external environment. For active sensing applications, the self-resonant frequency of antenna coil 166 with sensor 126 can range from 5 MHz to 50 MHz, and more specifically, from 10 MHz to 20 MHz.

In another example, sensor 126 can be incorporated into an inductor-resistor-capacitor (LCR) circuit, with antenna coil 166 as an inductor coil forming the inductor (L) and resistor (R) elements of the circuit, and sensor 126, connected in parallel, forming the capacitor (C) element. The LCR sensing circuit has a distinct self-resonant frequency, which can be represented as f=1/2π√LC(p), where L is the inductance of antenna coil 166 and C(p) is the capacitance of sensor 126 at a given parameter (e.g., pressure for a pressure sensor). The self-resonant frequency for an LCR sensing circuit can similarly range from 5 MHz to 50 MHz, and more specifically, from 10 MHz to 20 MHz.

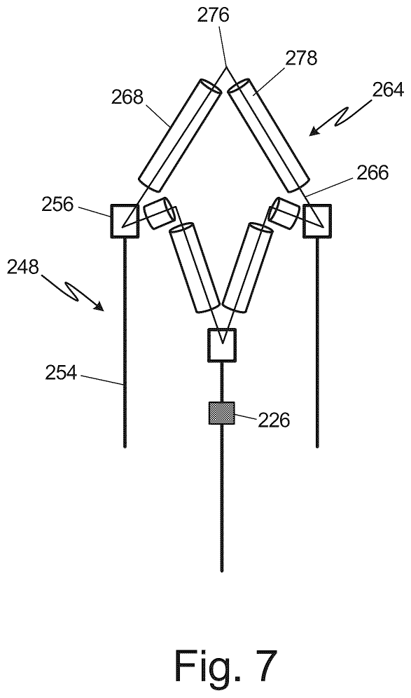

FIG. 6 is a simplified illustration of a second example of prosthetic heart valve 224, shown in the expanded state. FIG. 7 is a simplified illustration of post assemblies 248 and sensing circuit 264 of prosthetic heart valve 224 shown in the crimped state. FIGS. 6 and 7 are discussed together.

Prosthetic heart valve 224 is substantially similar to prosthetic heart valve 124, having deformable metallic frame 246 and three post assemblies 248 extending axially away from frame 246 at upper end 250. Lower end 252 is oppositely disposed from upper end 250 along axis A. Post assemblies 248 each include post 254 and islet 256. Frame 246 includes a plurality of interconnecting struts 258 defining cells 260 therebetween, with oppositely disposed points 262. Frame 246 is shown having a simplified design in FIG. 6, but can have the same design as frame 146 shown in FIG. 3. Prosthetic heart valve 224 further includes sensing circuit 264 having sensor 226 electrically connected to antenna coil 266. A plurality of hollow sleeves 268 circumscribe antenna coil 266. Antenna coil 266 can be secured to post assemblies 248 via sutures. Prosthetic heart valve 224 can be transitioned between the expanded and crimped states which alters its axial and radial dimensions as discussed above with respect to prosthetic heart valve 124.

Unlike the prior example, prosthetic heart valve 224 includes biocompatible fabric 274, configured as a skirt covering a portion of frame 246 on its exterior. In another example, fabric 274 can be differently disposed and/or cover the entire exterior of frame 246. Prosthetic heart valve 224 further differs from the previous example in that antenna coil 266 is configured as an angled square, or diamond shape in the expanded state. It is supported by three post assemblies 248 and extends primarily in the axial direction rather than being disposed mostly radially about upper end 250 of frame 246. More specifically, antenna coil 266 is configured with four corners 276 of uncovered antenna coil 266 and relatively straight sides 278 extending therebetween. Variously sized hollow sleeves 268 circumscribe antenna coil 266, with the two sides 278 furthest from frame 246 having a single long sleeve 268, and the two closest sides 278 each having two shorter sleeves 268. One corner 276 serves as the portion of antenna coil 266 closest to frame 246 (i.e., in the axial direction), while the opposite corner 276 is the portion of antenna coil 266 further from frame 246. The remaining two corners 276 are generally equidistant from frame 246. In the crimped state shown in FIG. 7, diamond shaped antenna coil 266 transitions to an arrowhead-shaped antenna coil 266. This occurs as the radially inward movement of prosthetic heart valve 224 causes the two closest sides 278 to bend between sleeve 268 to create two additional corners 276, such that the crimped, arrowhead-shaped antenna coil 266 has six corners 276. In the crimped state, the corner 276 closest to frame 246 and the corner 276 furthest from frame 246 are further apart (i.e., in the axial direction) than in the expanded state.

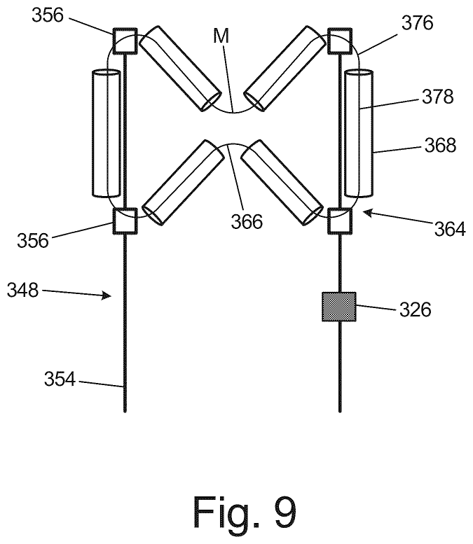

FIG. 8 is a simplified illustration of a third example of prosthetic heart valve 324, shown in the expanded state. FIG. 9 is a simplified illustration of sensing circuit 364 of prosthetic heart valve 324 shown in the crimped state. FIGS. 8 and 9 are discussed together.

Prosthetic heart valve 324 is substantially similar to prosthetic heart valves 124 and 224, having deformable metallic frame 346 and two post assemblies 348 extending axially away from frame 346 at upper end 350. Lower end 352 is oppositely disposed from upper end 350 along axis A. Each post assembly 348 includes extended post 354 with two islets 356. Frame 346 includes a plurality of interconnecting struts 358 defining cells 360 therebetween, with oppositely disposed points 362. Frame 346 is shown having a simplified design in FIG. 8, but can have the same design as frame 146 shown in FIG. 3. Biocompatible fabric 374 partially covers the exterior of frame 346. Prosthetic heart valve 324 further includes sensing circuit 364 having sensor 326 electrically connected to antenna coil 366. A plurality of hollow sleeves 368 circumscribe antenna coil 366. Antenna coil 366 can be secured to post assemblies 348 via sutures. Prosthetic heart valve 324 can be transitioned between the expanded and crimped states which alters its axial and radial dimensions as discussed above with respect to prosthetic heart valves 124 and 224.

Unlike the prior examples, antenna coil 366 is arranged as a rectangle, supported by two post assemblies 348 and extending primarily in the axial direction. Antenna coil 366 has four corners 376 aligned with gaps between adjacent sleeves 368. Extending between corners 376 are four sides 378, with two opposing sides 378 extending radially, and the remaining two opposing sides 378 extending axially. Each axially-extending side 378 can be circumscribed by one hollow sleeve 368, and each radially-extending side can be circumscribed by two hollow sleeves 368. Each radially-extending side 378 further includes a midpoint M located at a gap between adjacent sleeves 368. In the crimped state shown in FIG. 9, rectangular antenna coil 366 transitions to a butterfly-shaped antenna coil 366. This occurs as the radially inward movement of prosthetic heart valve 324 causes axially-extending sides 378 to move closer together, and midpoints M of radially-extending sides 378 to move toward one another such that portions of radially-extending sides 378 are angled between the respective midpoint M and axially-extending sides 378.

Antenna coils associated with prosthetic heart valves can experience detuning effects from the metallic frame. For each of the prosthetic heart valves discussed herein, the positioning of the respective antenna coils axially distant from the frame, with no direct physical contact with the frame has been shown in wireless detection testing to minimize the detuning effects of the frame without the presence of other detuning mitigation means (e.g., ferrite shielding layers). More specifically, an antenna coil axially offset about 1 cm from the frame may have a similar wireless detection range and resonance frequency to an antenna coil with no frame attached. With respect to antenna coil design, the majority of diamond-shaped antenna coil 266 (FIG. 6) is disposed further from frame 246, with the near corner 276 being the closest contact point. Such design may experience less detuning than rectangular antenna coil 366 (FIG. 8) with one radially-extending side 378 being closest to frame 346. Both designs may experience less detuning than antenna coil 166 (FIG. 3), as the entire length of antenna coil 166 is equidistant from frame 146. The deformable nature of the various antenna coils and sleeves further allows for crimping and re-expansion of the respective prosthetic heart valve with little to no change in self-resonance and no observed diminution of circuit performance.

Implantation of any of the prosthetic heart valves discussed herein inside the heart (e.g., heart 2) of a patient can include the following steps. First, a sterilized prosthetic heart valve is crimped using an appropriate crimping tool so that the prosthetic heart valve can be inserted into the delivery vehicle (e.g., expandable catheter). The crimped prosthetic heart valve can be inserted into the delivery site (e.g., aortic valve 18), and once properly positioned, can be expanded (e.g., by expanding the catheter) into the surrounding tissue, and the delivery vehicle removed. The dimensions of the pre-crimping expanded state and the final expanded state of the prosthetic heart valve can be substantially similar in one example. In an alternative example, the final expanded state of the prosthetic heart valve can be different (e.g., smaller) than the pre-crimping expanded state.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Discussion of Detailed Embodiments

The following are non-exclusive descriptions of possible embodiments of the present invention.

A prosthetic valve includes a flexible frame disposed along and deformable about a frame axis between a crimped state and an expanded state. The frame includes a first end, a second end oppositely disposed from the first end, a plurality of post assemblies extending axially away from the first end, and a network of interconnected struts defining a plurality of cells. A circuit is disposed axially away from the frame and mounted on the plurality of post assemblies. The circuit includes an antenna coil secured to at least two post assemblies of the plurality of post assemblies, and a sensor in electrical communication with the antenna coil. The sensor is configured to sense a physical parameter and generate a signal representing the physical parameter.

The prosthetic valve of the preceding paragraph can optionally include, additionally and/or alternatively, any one or more of the following features, configurations and/or additional components:

The prosthetic valve further includes a plurality of hollow sleeves circumscribing the antenna coil.

Wherein the plurality of post assemblies consists of four post assemblies.

Wherein in the expanded state, the antenna coil has a circular shape in a radial direction.

Wherein in the crimped state, the antenna coil has a zig-zag shape in an axial direction, having a plurality of peaks and troughs.

Wherein the plurality of post assemblies consists of three post assemblies.

Wherein in the expanded state, the antenna coil has a diamond shape with a first corner disposed a further axial distance from the frame than an oppositely disposed second corner.

Wherein in the crimped state, the antenna coil has an arrowhead shape, and wherein an axial distance between the frame and the first corner is greater than in the expanded state.

Wherein the plurality of post assemblies consists of two post assemblies.

Wherein in the expanded state, the antenna coil has a rectangular shape with a first pair of opposing axially-extending segments, and a second pair of opposing radially-extending segments.

Wherein in the crimped state, the antenna coil has a butterfly shape with a midpoint of each of the opposing radially-extending segments disposed closer together than when in the expanded state.

Wherein the antenna coil is formed from gold.

Wherein the antenna coil is further coated with an insulating material.

Wherein the frame is formed from a biocompatible metallic material.

Wherein each post assembly of the plurality of post assemblies comprises a post and at least one islet.

Wherein the sensor is mounted on one post assembly of the plurality of post assemblies.

Wherein the sensor is a capacitive pressure sensor, and wherein the sensed physical parameter is pressure.

Wherein the circuit has a self-resonant frequency ranging from 5 MHz to 50 MHz. Wherein the self-resonant frequency ranges from 10 MHz to 20 MHz.

Wherein the prosthetic valve is implantable in a heart valve of a patient.

Wherein in an implanted state of the prosthetic valve, the axis of the frame aligns with a flow of blood through the prosthetic valve.

Wherein the prosthetic valve is deliverable to the heart valve of the patient via an expandable catheter.

Wherein the prosthetic valve is sterilized.

A method of implanting the prosthetic valve in an organ of a patient includes transitioning the prosthetic valve from the expanded state to the crimped state, delivering, via an expandable catheter, the prosthetic valve into the organ while in the crimped state, and returning the prosthetic valve to the expanded state once inside the organ of the patient by expanding the expandable catheter.

Wherein in the crimped state, the frame has a first axial dimension and a first radial dimension.

Wherein in the expanded state, the frame has a second axial dimension and a second radial dimension.

Wherein the first axial dimension is greater than the second axial dimension, and the first radial dimension is smaller than the second radial dimension.

The method further includes sterilizing the prosthetic valve prior to delivering the prosthetic valve to the organ.

The method further includes removing the expandable catheter from the patient after returning the prosthetic valve to the expanded state.

A prosthetic valve includes a flexible frame disposed along and deformable about a frame axis between a crimped state and an expanded state. The frame includes a first end, a second end oppositely disposed from the first end, a plurality of post assemblies extending axially away from the first end, and a network of interconnected struts defining a plurality of cells. A circuit is disposed axially away from the frame and mounted on the plurality of post assemblies. The circuit includes an antenna coil secured to at least two post assemblies of the plurality of post assemblies, and a sensor in electrical communication with the antenna coil. A plurality of hollows sleeves circumscribe the antenna coil. The sensor is configured to sense a physical parameter and generate a signal representing the physical parameter.

The prosthetic valve of the preceding paragraph can optionally include, additionally and/or alternatively, any one or more of the following features, configurations and/or additional components:

Wherein the plurality of post assemblies consists of four post assemblies.

Wherein in the expanded state, the antenna coil has a circular shape in a radial direction.

Wherein in the crimped state, the antenna coil has a zig-zag shape in an axial direction, having a plurality of peaks and troughs.

Wherein the plurality of post assemblies consists of three post assemblies.

Wherein in the expanded state, the antenna coil has a diamond shape with a first corner disposed a further axial distance from the frame than an oppositely disposed second corner.

Wherein in the crimped state, the antenna coil has an arrowhead shape, and wherein an axial distance between the frame and the first corner is greater than in the expanded state.

Wherein the plurality of post assemblies consists of two post assemblies.

Wherein in the expanded state, the antenna coil has a rectangular shape with a first pair of opposing axially-extending segments, and a second pair of opposing radially-extending segments.

Wherein in the crimped state, the antenna coil has a butterfly shape with a midpoint of each of the opposing radially-extending segments disposed closer together than when in the expanded state.

Wherein the antenna coil is formed from gold.

Wherein the antenna coil is further coated with an insulating material.

Wherein the frame is formed from a biocompatible metallic material.

Wherein each post assembly of the plurality of post assemblies comprises a post and at least one islet.

Wherein the sensor is mounted on one post assembly of the plurality of post assemblies.

Wherein the sensor is a capacitive pressure sensor, and wherein the sensed physical parameter is pressure.

Wherein the circuit has a self-resonant frequency ranging from 5 MHz to 50 MHz.

Wherein the self-resonant frequency ranges from 10 MHz to 20 MHz.

Wherein the prosthetic valve is implantable in a heart valve of a patient.

Wherein in an implanted state of the prosthetic valve, the axis of the frame aligns with a flow of blood through the prosthetic valve.

Wherein the prosthetic valve is deliverable to the heart valve of the patient via an expandable catheter.

Wherein the prosthetic valve is sterilized.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A prosthetic valve comprising:
a flexible frame disposed along a frame axis and deformable about the frame axis between a crimped state and an expanded state, the frame comprising:
a first end;
a second end oppositely disposed from the first end;
a plurality of post assemblies extending axially away from the first end; and
a network of interconnected struts defining a plurality of cells;
a circuit disposed axially away from the frame and mounted on the plurality of post assemblies, the circuit comprising:
an antenna coil secured to at least two post assemblies of the plurality of post assemblies; and
a sensor in electrical communication with the antenna coil; and
a plurality of hollow sleeves circumscribing the antenna coil;
wherein the sensor is configured to sense a physical parameter and generate a signal representing the physical parameter.

2. The prosthetic valve of claim 1, wherein the plurality of post assemblies consists of four post assemblies.

3. The prosthetic valve of claim 2, wherein in the expanded state, the antenna coil has a circular shape in a radial direction, and wherein in the crimped state, the antenna coil has a zig-zag shape in an axial direction, having a plurality of peaks and troughs.

4. The prosthetic valve of claim 1, wherein the plurality of post assemblies consists of three post assemblies.

5. The prosthetic valve of claim 4, wherein in the expanded state, the antenna coil has a diamond shape with a first corner disposed a further axial distance from the frame than an oppositely disposed second corner, and wherein in the crimped state, the antenna coil has an arrowhead shape, and wherein an axial distance between the frame and the first corner is greater than in the expanded state.

6. The prosthetic valve of claim 1, wherein the plurality of post assemblies consists of two post assemblies.

7. The prosthetic valve of claim 6, wherein in the expanded state, the antenna coil has a rectangular shape with a first pair of opposing axially-extending segments, and a second pair of opposing radially-extending segments, and wherein in the crimped state, the antenna coil has a butterfly shape with a midpoint of each of the opposing radially-extending segments disposed closer together than when in the expanded state.

8. The prosthetic valve of claim 1, wherein the antenna coil is formed from gold.

9. The prosthetic valve of claim 8, wherein the antenna coil is further coated with an insulating material.

10. The prosthetic valve of claim 1, wherein the frame is formed from a biocompatible metallic material.

11. A prosthetic valve comprising:
a flexible frame disposed along a frame axis and deformable about the frame axis between a crimped state and an expanded state, the frame comprising:
a first end;
a second end oppositely disposed from the first end;
a plurality of post assemblies extending axially away from the first end, wherein each post assembly of the plurality of post assemblies comprises a post and at least one islet; and
a network of interconnected struts defining a plurality of cells;
a circuit disposed axially away from the frame and mounted on the plurality of post assemblies, the circuit comprising:
an antenna coil secured to at least two post assemblies of the plurality of post assemblies; and
a sensor in electrical communication with the antenna coil; and
wherein the sensor is configured to sense a physical parameter and generate a signal representing the physical parameter.

12. The prosthetic valve of claim 11, and further comprising:
a plurality of hollow sleeves circumscribing the antenna coil.

13. The prosthetic valve of claim 11, wherein the antenna coil is threaded through the at least one islet.

14. The prosthetic valve of claim 11, wherein the sensor is mounted on one post assembly of the plurality of post assemblies.

15. The prosthetic valve of claim 14, wherein the sensor is a capacitive pressure sensor, and wherein the sensed physical parameter is pressure.

16. The prosthetic valve of claim 11, wherein the prosthetic valve is implantable in a heart valve of a patient, and wherein in an implanted state of the prosthetic valve, the axis of the frame aligns with a flow of blood through the prosthetic valve.

17. A prosthetic valve comprising:
a flexible frame disposed along a frame axis and deformable about the frame axis between a crimped state and an expanded state, the frame comprising:
a first end;
a second end oppositely disposed from the first end;
a plurality of post assemblies extending axially away from the first end; and
a network of interconnected struts defining a plurality of cells;
a circuit disposed axially away from the frame and mounted on the plurality of post assemblies, the circuit comprising:
an antenna coil secured to at least two post assemblies of the plurality of post assemblies; and
a sensor in electrical communication with the antenna coil; and
wherein the sensor is configured to sense a physical parameter and generate a signal representing the physical parameter; and
wherein the circuit has a self-resonant frequency ranging from 5 MHz to 50 MHz.

18. The prosthetic valve of claim 17, and further comprising:
a plurality of hollow sleeves circumscribing the antenna coil.

19. The prosthetic valve of claim 17, wherein the self-resonant frequency ranges from 10 MHz to 20 MHz.

20. The prosthetic valve of claim 17, wherein the circuit is an active sensing circuit.
